(19) United States
(12) Patent Application Publication
(10) Pub. No.: US 2026/0053659 A1
(43) Pub. Date: Feb. 26, 2026

(54) SYSTEM FOR DETERMINING WEAR TIME OF CRANIAL REMODELING ORTHOSIS DEVICE

(71) Applicant: CRANIAL TECHNOLOGIES, INC., TEMPE, AZ (US)

(72) Inventors: TIMOTHY R LITTLEFIELD, MESA, AZ (US); JEROLD N LUISI, PHOENIX, AZ (US); ANNA LEIGH TREBILCOCK, CASA GRANDE, AZ (US)

(73) Assignee: CRANIAL TECHNOLOGIES, INC., TEMPE, AZ (US)

(21) Appl. No.: 18/813,801

(22) Filed: Aug. 23, 2024

(51) Int. Cl. A61F 5/058; G16H 20/00

(52) U.S. Cl. CPC A61F 5/05891; G16H 20/00

(57) ABSTRACT

A system for determining cranial remodeling orthosis (CRO) device wearing time comprises a CRO device comprising at least one sensor device having a sensor memory. The system further comprises an electronic device external to the CRO device. The at least one sensor device is operable to capture specific condition parameters and to store corresponding parameter data in the sensor memory at periodic intervals. The parameter data stored in the sensor memory may be accessed and the system further comprises a software program stored in a non-transient memory and comprising instructions executable to analyze the parameter data to determine wear times for the CRO device.

### BACKGROUND

Thousands of infants are born with cranial deformities every year. Treatment of cranial deformities in infants through a treatment program utilizing cranial remodeling orthosis devices is well known. Cranial remodeling orthosis (“CRO”) devices shape the infant's head as it grows by restraining growth in some areas of the cranium and permitting growth in other areas. Treatment programs utilizing CRO devices are highly effective.

The Applicant of this invention has pioneered development and use of CRO devices that are custom made for each infant patient because each infant's cranial deformity is unique. The Applicant of this invention has developed pioneering methodologies of manufacturing custom made CRO devices that along with its treatment programs are highly effective. Applicant was the first to obtain U.S. Federal Drug Administration clearances for its custom made CRO devices for both deformational (i.e., non-synostotic) plagiocephaly as well as for post-operative applications, as evidenced by a 510 k issued to Applicant.

Treatment plans for correction of infant cranial deformities with cranial remodeling orthosis devices typically require that the CRO device be worn in accordance with a treatment wearing schedule. The wearing schedule typically requires wearing the device daily for an extended period of time, typically twenty-three hours on and one hour off.

Frequently, parents are not aware that the wearing time is not being followed or the consequences of not complying with wearing schedule.

Treatment specialists providing cranial remodeling orthosis devices have a need to verify that infant patients are complying with orthotic wear schedules to ensure that treatments are being properly followed.

The most consequential result of failure to wear the CRO device in accordance with the wearing schedule which may then lead to an extended time for such treatment and/or requiring the use an additional custom CRO device.

In addition, failure to wear the device on the prescribed schedule may also lead to fit issues which result in the product needing to be remade for the patient if they are to continue treatment.

In addition, failure to comply with the wearing schedule can lead to patient's parents questioning the effectiveness of the treatment, resulting in complaints, demands for additional CRO devices, or demands for refunds of the payments for treatment.

Accordingly, it is desirable to provide a method and system to objectively determine wearing compliance of CRO devices in accordance with prescribed wearing schedules.

### SUMMARY

An embodiment of a CRO device comprises an outer portion comprising an outer surface and an inner surface and an inner layer carried on the outer portion inner surface and having an inner surface adapted to contact the head of a patient in first predetermined areas and spaced apart from the head of the patient in other predetermined areas. A sensor is carried by the CRO device. The sensor comprises a sensor memory. The sensor may comprise a unique sensor identification code associated therewith and readable by the electronic device. The sensor is operable to capture specific condition parameters and to store corresponding parameter data in the sensor memory at periodic intervals. The parameter data stored in the sensor memory is readable by an external electronic device. The parameter data is utilizable by a processor to determine wear time periods for the CRO device.

An embodiment of a system for determining CRO device wearing time comprises a CRO device comprising at least one sensor device. The sensor device comprises a sensor memory. The system further comprises an electronic device external to the CRO device. The at least one sensor device is operable to capture specific condition parameters and to store corresponding parameter data in the sensor memory at periodic intervals. The electronic device comprises an interface for communicating with the at least one sensor to access the parameter data stored in the sensor memory. The system further comprises a software program stored in a non-transient memory and comprising instructions executable to analyze the parameter data to determine wear times for the CRO device.

### BRIEF DESCRIPTION OF THE DRAWING

The invention will be better understood from a reading of the following detailed description in conjunction with the drawing in which like reference designators are used to identify like elements, and in which:

FIG. 1 is a front isometric view of a CRO device;

FIG. 2 is a side isometric view of a CRO device;

FIG. 3 is a rear isometric view of a CRO device

FIG. 4 is an isometric view of an embodiment or a sensor;

FIG. 5 is a block diagram of the sensor of FIG. 4;

FIG. 6 illustrates a CRO system;

FIG. 7 is a graph of sensor readings obtained by the system of FIG. 6;

FIG. 8 shows a mobile device display screen;

FIGS. 9-14 are cross-sections of a portion of a CRO device;

FIG. 15 is a block diagram of a second sensor;

FIG. 16 is a flow chart; and

FIG. 17 is a flow chart.

### DETAILED DESCRIPTION

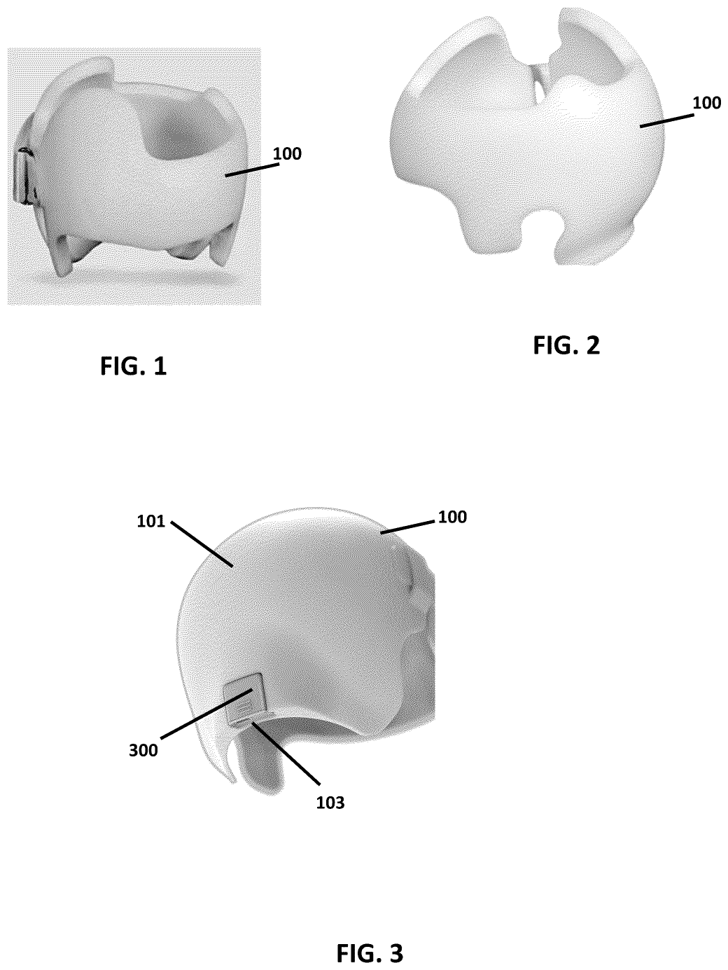

FIGS. 1 and 2 illustrate an exemplary CRO device 100 that has been custom manufactured for an infant patient. It will be appreciated by those skilled in the art that the configuration of CRO device 100 may be different for treatment of different patient cranial deformities and that the inventions described herein are not limited to any particular CRO device configuration.

CRO device 100 corrects cranial deformity by taking advantage of the rapid growth of an infant's cranium by guiding that growth. Growth is guided by contacting the infant's head at certain areas to restrict growth in those areas and by allowing the infant's head to grow in other areas that are not contacted.

CRO devices are to be worn for a predetermined time period daily. Typically, the wear time is 23 hours on and one hour off. With this wearing schedule, the duration of treatment with a CRO device is minimized and treatment is most effective.

After a patient has had a custom CRO device manufactured, the patient is fitted with the CRO device and instructions are given including wear time. Subsequently, the patient is seen every two weeks to check on treatment progress.

Compliance with the wearing schedule is important to effectiveness of the treatment.

One benefit of knowing CRO device wearing times is that it can be determined whether the CRO device is being worn in compliance with prescribed wear schedules. Compliance with prescribed wear schedules produces better CRO treatment outcomes with the result that treatment times are shorter and fewer follow-up appointments with a clinician or physician is necessary as compared to instances where wearing compliance is uncertain.

Various embodiments of systems that are utilized to determine CRO device wearing times are described herein. In the various embodiments described herein, one or more sensors are integrated into each CRO device.

A cranial remodeling orthosis (CRO) device comprises an outer portion comprising an outer surface and an inner surface and an inner layer carried on the outer portion inner surface and having an inner surface adapted to contact the head of a patient in first predetermined areas and spaced apart from the head of said patient in other predetermined areas.

In one embodiment, a sensor is carried on the interior of the CRO device. The sensor comprises a sensor memory. The sensor being is activatable by an electronic device external to the CRO device. The sensor comprises a unique sensor identification code associated therewith and readable by the electronic device.

The sensor, after activation, is operable to capture specific condition parameters and to store corresponding parameter data in the sensor memory at periodic intervals. The parameter data stored in the sensor memory is readable by the external electronic device and is utilizable by a processor to determine wear time periods for the CRO device being worn by a patient.

The sensor may comprise an interface accessible by the external electronic device to read temperature data from memory.

The memory may store a predetermined number of parameter data. The sensor device overwrites oldest stored temperature data when more than the predetermined number of parameter data is to be stored in memory.

In other embodiments, the memory is sized large enough to store all parameter data during a course of treatment.

In one embodiment, the sensor is carried on the CRO device outer portion inner surface.

In one embodiment the CRO device comprises a plurality of apertures extending through the inner layer from the Inner layer inner surface to the sensor.

In another embodiment the CRO device comprises a channel in the inner layer extending to the inner layer inner surface, the channel carrying the sensor therein.

The channel extends to the first portion inner surface and carries a thermal conductive gel.

In one embodiment, the sensor is carried on the inner layer inner surface.

In various embodiments, the sensor is a temperature sensor.

In various embodiments, the sensor comprises a plurality of sensing elements disposed in different positions on the CRO device, and the plurality of sensing elements capture a corresponding plurality of specific condition parameters simultaneously store a corresponding plurality of parameter data in memory.

In certain embodiments one first sensing element of the plurality of sensing elements captures first temperature condition parameters proximate the outside surface of the CRO device outer portion, and a second sensing element captures second temperature condition parameters proximate the inner surface of the CRO device inner layer.

In certain embodiments, the parameter data comprises a differential between each first temperature condition parameter and each corresponding second temperature condition parameter.

In other embodiments, the parameter data comprises first temperature condition parameter data and said second temperature condition parameter data.

In one embodiment, one first sensing element captures first temperature condition parameters proximate said inner surface of said outer portion, and a second sensing element of captures second condition parameters. The parameter data comprises the first temperature condition parameter data and the second condition parameter data.

In various embodiments, the periodic sampling intervals are selectable.

FIG. 3 shows representative CRO device 100 with a representative sensor 300 carried thereon. It will be understood by those skilled in the art that the physical configuration of sensor 300 shown in the drawing Figures is not intended in any way to be limiting to the invention as claimed.

Sensor 300 is carried by CRO device 100 in a receptacle 103 formed in outer shell 101.

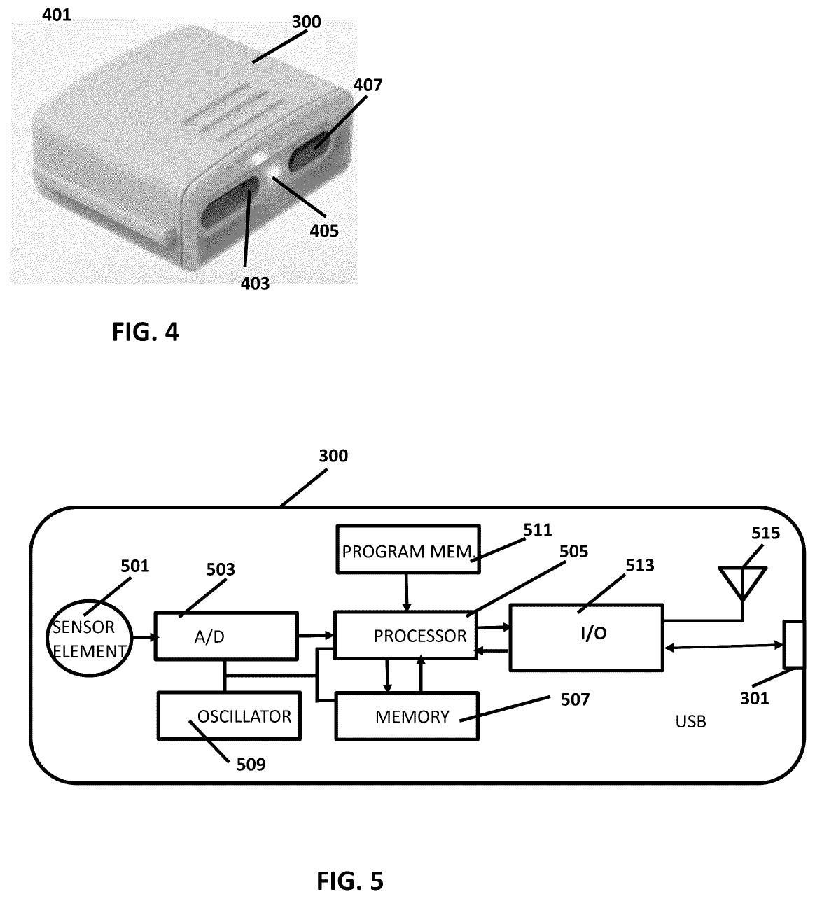

Sensor 300 shown in FIG. 4. includes a USB port 301 for transferring data and for charging a sensor rechargeable battery. In other embodiments a different type of connector may be used for transferring data and/or for charging a sensor rechargeable battery. In other embodiments the battery may be of a non-rechargeable type and after the battery is exhausted, sensor 300 will be non-functional.

Data may also be transferred via a wireless connection to a device. By way of non-limiting example, Bluetooth or WiFi or near field connection may be used to transfer data. In other embodiments, USB port 301 may not be included in sensor 300.

Sensor 300 may also include a light emitting diode 303 to provide visual feedback such as on/off status, battery level and other indications. Sensor 300 may further include a switch 305 utilizable to control on/off power and status control.

In other embodiments, sensor 300 may not have a light emitting diode 303 or other visual indicator and in other embodiments, sensor 300 may not have a switch.

In other embodiments, sensor 300 may be reprogrammable. For example, program memory 511 and/or memory 507 may be programmed and or reallocated, and/or the sampling frequency may be changed, and/or threshold values may be changed, and/or various latent features may be activated to improve precision of sensor 300.

In other embodiments, all access to sensor 300 may be via a wireless connection.

Each sensor 300 has a unique identification code associated with it. In the embodiment shown, the unique identification code is stored in sensor 300 and is readable via USB port 301 or via a wireless connection, e.g. blue tooth.

Each CRO device also has a unique code associated with it so that sensor 300, when installed on CRO device 100 and activated, is associated with a specific CRO device.

Turning now to FIG. 5, a representative embodiment of the structure of sensor 300 is shown. Sensor 300 comprises a sensing element 501 that, when activated, generates an output signal that is representative of the parameter being sensed. In this embodiment sensing element 501 generates an analog output signal that is applied to analog to digital (A/D) circuit 503. A/D circuit 530 samples the analog output signal at periodic sampling intervals and converts the analog signal to digital representations of the sensed parameters. The periodic sampling intervals or sampling rate is determined by processing circuitry or processor 509 utilizing oscillator 509. Processor 505 stores digital representations of samples in memory 507. The sampling rate may be programmable in various embodiments.

Memory 507 is sized to store periodic samples accumulated over a predetermined period of time. By way of example, memory 507 may be sized to stores samples captured every 5 to 15 minutes for a two or three week period. It will be recognized that the size of memory 507 may be different in different embodiments. For example, memory 507 may be of such a size as to store all samples taken throughout a course of treatment. Memory 507 may be configured such that when the memory 507 is filled, the oldest entries are overwritten with the newest entries so that the memory always contains the most recent samples.

In addition to memory 507, sensor 300 comprises a read-only memory (ROM) that is programmed to contain the unique sensor identification code.

Sensor 300 further comprises input/output (I/O) circuitry 513 coupled to USB port 301 and to an internal antenna 515 for wireless access. In other embodiments only one of a USB port 301 or internal antenna 515 may be provided.

In various embodiments, sensor 300 may include a digital clock or counter that provides a time marker when a sensed parameter is captured and stored in memory 507. The marker may also be stored with the sensed parameter. In other embodiments the sampling period, i.e., the time between samples may be used to calculate the time or relative time between samples in combination with the activation time of sensor 300 and memory space in memory 507 is not needed to store the time of sensed parameter capture.

By way of example, if the sampling interval is selected to be 5 minutes, every 12 intervals is equal to one hour.

If memory 507 stores one parameter sample every 5 minutes and the maximum time between clinician checkups is three weeks, the number of memory locations in memory 507 that are needed to store the sampled parameters is 12 samples per hour×24 hours per day×21 days in three weeks=6048. Accordingly, memory 507 would need 6048 locations to store the sampled parameters and the memory location would be an indicator of the time after sensor activation.

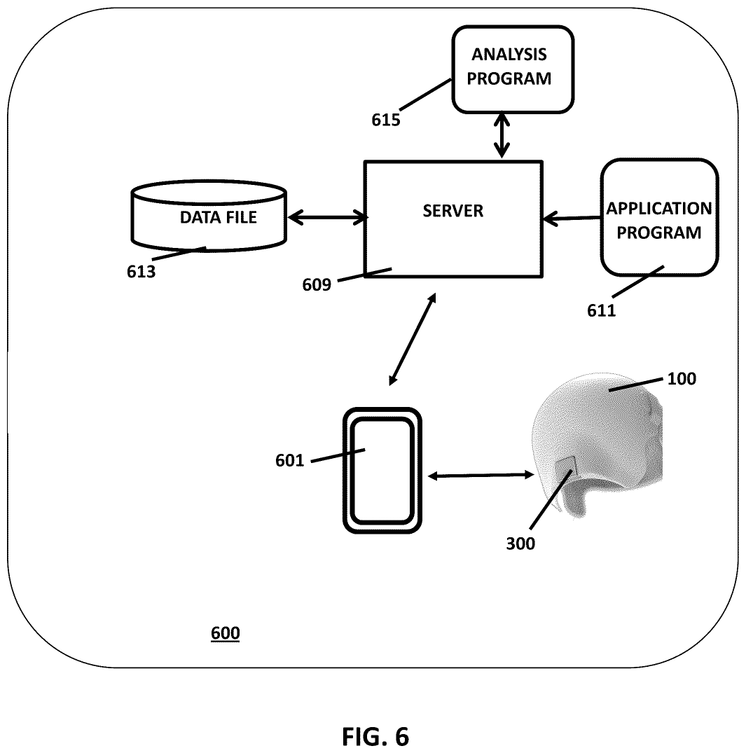

As shown in FIG. 6, sensor 300 is part of a system 600 that advantageously determines wear time on a daily basis for CRO device 100. The operation of system 600 is such that the samples stored in sensor 300 are accessed and processed to determine daily wear times of CRO device 100.

In this embodiment, a mobile device 601 such as a smart phone or tablet computer is utilized to access the samples from sensor 300.

Access to an application program 611 available via server 609 is restricted to authorized users of system 600, typically a clinician treating an infant patient with CRO device 100. The clinician having authorization utilizes mobile device 601 to download application program 611 from server 609.

Application program 611 is such that it provides a graphical user interface (GUI) to the user of device 601. The GUI provides step-by-step instructions to the clinician. The clinician is instructed to access the unique identification code for CRO device 100. Mobile device 601 is used to access the unique identification code for CRO device 100. The code may be carried on CRO device 100 as a bar code or other visual code readable by mobile device 600. Alternatively, CRO device may have a radio frequency (rf) identification chip that is readable by a wireless methodology by CRO device 100. Still further, CRO device may carry a visually readable unique identification code that is manually entered into mobile device 601 by the clinician.

After entering the CRO device unique identification code, the clinician is instructed to obtain the sensor unique identification code. For embodiments comprising a USB port the clinician may connect mobile device 600 to USB port 403 to access the sensor unique identification code. For embodiments without a USB port 403, mobile device 600 wirelessly accesses sensor 300 using a Bluetooth connection.

The clinician is instructed to enter patient specific information. Mobile device 600, executing application program 611, associates the patient specific information with the unique sensor identification code and the unique CRO device identification code.

After entering the patient specific information and obtaining the sensor unique identification code and unique CRO device identification code, mobile device 600 executing application program 611 is used to activate sensor 300 and to upload the patient specific information and the unique sensor and CRO device identification codes to server 609. Uploading of patient specific information and the unique sensor and CRO device codes is done using a WiFi connection by mobile device 600 via the Internet to server 609.

In other embodiments, patient specific information and the unique sensor and CRO device codes may be stored in mobile device 600.

Upon receiving patient specific information and the associated sensor and CRO device identification codes, server 609 creates a patient file in memory 613.

Upon activation, sensor 300 starts periodic sampling of sensed parameters and storing the sensed parameter samples in memory 507.

At the next scheduled appointment for the clinician to see the patient, typically in two weeks after the initial fitting of CRO device 100, the clinician utilizes mobile device 601 to access sensor 300 via Bluetooth to access the unique sensor identification and the unique CRO device identification. Mobile device 601 uploads stored samples of sensed parameters from sensor memory 507 in sensor 300.

In the embodiment of FIG. 6, mobile device 601 forwards the uploaded stored samples to server 609. Server 609 stores the uploaded stored samples in data file 613 associated with the unique sensor identification and the unique CRO device identification.

Server 609 executes an analysis program 615 to operate on the stored samples. Analysis program 615 executes instructions stored in a non-transient memory to operate on the stored samples. The executed instructions utilize an algorithm to analyze the sensed parameters to determine the wearing time of CRO device 100.

In one embodiment, sensor 300 is a temperature sensor that senses temperature and stores the sensed temperature as the parameters in sensor 300.

Analysis program 615 uses an algorithm to operate on sensed parameters to determine when CRO device 100 is being worn. Analysis program 615 analyzes the sensed parameters and identifies times that can be considered times when CRO device 100 is placed on a head and times when CRO device 100 is removed from the head.

In one embodiment, analysis program 615 utilizes one or more algorithms to determine from the sensed parameters when specific first characteristic changes in the sensed parameters occur to determine when CRO device 100 is placed on a head and when specific second characteristic changes in the sensed parameters occur to determine when CRO device 100 is removed from the head. By knowing when CRO device 100 is placed on a head and removed from the head, a wear time of CRO device 100 is determined.

In the embodiment utilizing a temperature sensing element in sensor 300 Analysis program 615 analyzes sensed data to determine when the sensed temperature rises in a predetermined manner and when it falls in a predetermined manner. By utilizing analysis program 615, each instance can be determined when the sensed temperature begins to rise and each instance when the temperature begins to fall.

In experiments, we have determined that the reaction rate of sensor 300, when exposed to two known temperature extremes, i.e., contact with the head and noncontact exposure to ambient environment, is sufficient to be able to determine wear times.

The very sharp transition down, once removed from the heat source i.e., the body and exposure to an ambient environment suggests that there may be at least two approaches to determine periods of noncompliance.

One way would be setting a predetermined threshold value and determining when this value is reached or crossed. From tests, it would appear that a value below the predetermined threshold value would indicate that the band was not being worn, but rather was sitting unused in an ambient environment.

Another approach is to identify the inflections of the temperature curve for rapid changes in the slope of the data. This is a very robust way to observe when there is a sudden change in what the sensor is exposed to, i.e., wearing and removal of CRO device 100.

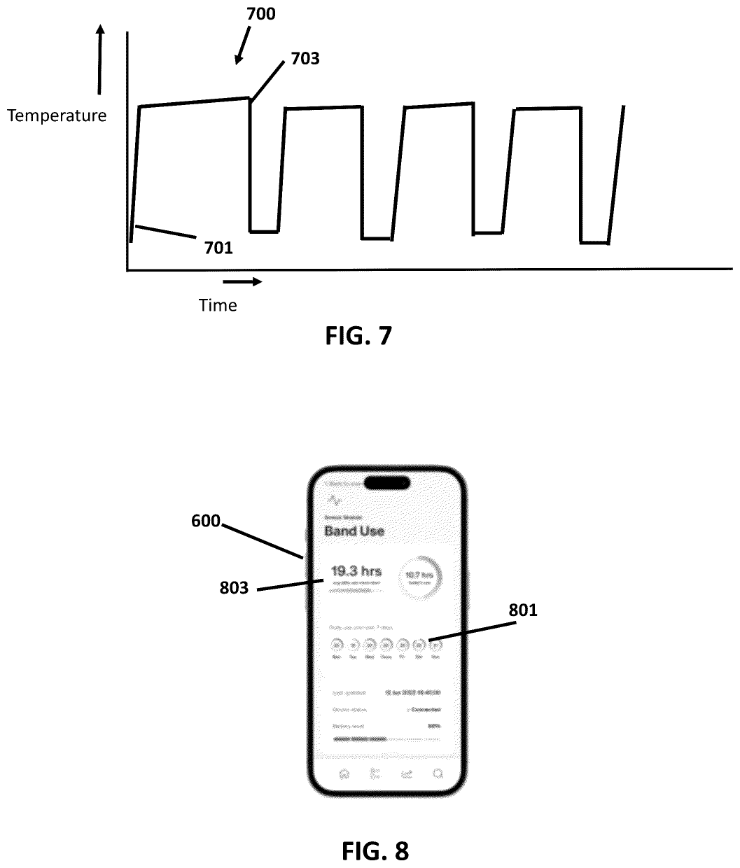

By way of illustrative example, reference is directed to FIG. 7 that illustrates a plot 700 of sampled temperature data for a four-day period. Each time CRO device 100 is placed on the patient's head, the temperature sensed by sensor 300 beings to rise from ambient temperature as shown by curvature point 701, approaching the head temperature of the patient as shown by curvature portion 703. Each time CRO device 100 is removed from the head of the patient, the temperature sensed by sensor 300 begins to decrease towards ambient temperature as shown by curvature point 701. The times that CRO device 100 is being worn by the patient can be determined from determining each moment that the temperature begins to rise from ambient temperature until the immediate next moment that the temperature begins to fall.

In other embodiments, analysis program 615 may utilize minimum and maximum measured temperatures to interpret data as indicating that CRO device 100 is off and on.

In other embodiments, analysis program 615 may utilize the rates of change of measured temperatures, derivatives of the temperature, and inflection points in the measured temperatures.

As pointed out above, a clinician typically uploads sensed parameters for a two week or longer interval. Accordingly, the curve of sensed parameters would include data for the period of time that is uploaded. All the sensed data for a patient is stored in data file 613 and can be retrieved by the clinician. In addition, the plot 700 for a patient may be retrieved for any time period that has been uploaded.

As described herein above, analysis program 615, when executed by processor 609 also provides a GUI for display on mobile device 601. The GUI is operable to display the curve of most recently sensed data on mobile device 601. In addition, the GUI may be utilized to display the curve of sensed parameters on a day-by-day basis with corresponding calculated wear times on a day-by-day basis.

FIG. 8 illustrates one GUI displayed on a smartphone 600. The GUI display in this embodiment shows the wear time of CRO device 100 on a day-by-day basis 601 and for a selected day 603. It will be appreciated by those skilled in the art that various other GUI displays may be provided. In one embodiment, the GUI display may show only those days in which the prescribed wear time is not met.

One embodiment of a system for determining CRO device wearing time comprises a CRO device; comprising at least one sensor device, said sensor device comprising a sensor memory. An electronic device is external to the CRO device. The at least one sensor device is responsive to the electronic device to become activated. After being activated, the sensor device is operable to capture specific condition parameters and to store corresponding parameter data in the sensor memory at periodic intervals. The electronic device comprises an interface for communicating with the at least one sensor to access the parameter data stored in the sensor memory. The system further comprises a software program stored in a non-transient memory and comprising instructions executable to analyze the parameter data to determine wear times for the CRO device.

In an embodiment of the system, the software program utilizes one or more algorithms to determine from parameter data when specific first characteristic changes in parameter data occur to determine when the CRO device is worn on a head and when specific second characteristic changes in parameter data occur to determine when the CRO device is removed from the head.

In one embodiment, the software program operates on parameter data to identify reaction rates of the sensor device to determine wear times of the CRO device.

In various embodiments, the software program executes instructions to determine when parameter data reaches predetermined threshold values to determine wear times of the CRO device.

In embodiments, the software program executes instructions to determine rapid changes in a slope of parameter data to determine wear times of the CRO device.

In certain embodiments, the software program executes instructions to identify changes in parameter data derivatives and inflection points in parameter data to determine wear times.

In various embodiments, the sensor device comprises a temperature sensor positioned on the CRO device. The specific condition parameters are temperatures within the CRO device and the parameter data comprises captured temperatures.

In various embodiments, the sensor device comprises a unique sensor identification code; and the electronic device is operable to read the unique sensor identification code.

In several embodiments the CRO device comprises a unique CRO device identification code and the electronic device may be operable to read the unique CRO device identification code.

In several embodiments, the sensor device comprises a temperature sensor.

In several embodiments, the electronic device comprises one of a smart phone, tablet, and mobile computer.

The electronic device may comprise a set of executable instructions operable to activate the sensor when the cranial remodeling orthosis is to be initially provided to a patient.

In various embodiments, the system comprises a server comprising the processor, and the electronic device is in electronic communication with the processor.

In various embodiments, the electronic device comprises a graphical user interface (GUI)
that selectively displays wear time for the CRO device.

There are different methodologies that may be used to manufacture CRO device. Typically, a CRO device 100, shown in cross-section in FIGS. 8 through 13, inclusive comprises an outer shell 803, that may be manufactured utilizing additive manufacture or otherwise, and one or more inner layer 805 that may be manufactured by additive manufacture or by other manufacturing technique. Inner layer 805 may be a liner of a type conventionally utilized in CRO devices and may comprise Pelite or other foam or cellular material or another liner.

In various embodiments, sensor 300 may be in contact with the patient's head or not. Sensor 300 may be on the inner surface of CRO device 100 or recessed into the inner layer or disposed below the inner layer, i.e., sandwiched between a foam inner layer an outer layer. Sensor 300 may be recessed from into the inner layer to minimize insulative effects. The inner layer may have holes or apertures to allow better heat transmission to sensor 300. In other embodiments, a more thermally conductive medium such as a thermal gel may be provided between sensor 300 and the patient's head.

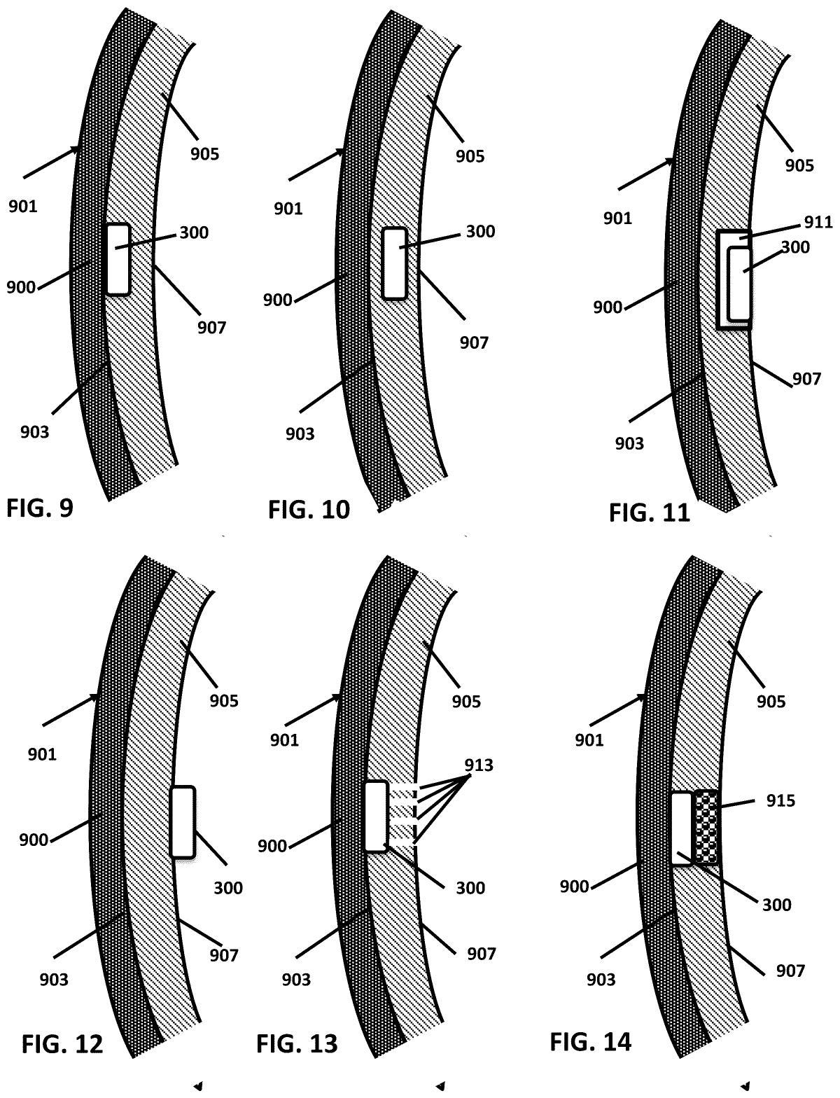

Each of FIGS. 9-14 illustrates a cross-section of an additional embodiment of CRO device 100 showing a different placement of a sensor 300. In each of the embodiments of FIGS. 9-14 the CRO device comprises an outer layer 900 having an outer surface 901 and an inner surface 903. Inner surface 903 carries an inner layer 905 having an inner surface 907 that is in an area that contacts the patient's head when CRO device 100 is worn. Inner layer 905 in the embodiments shown comprises Pelite, but in other embodiments may comprise other material.

In the embodiment of FIG. 9, sensor 300 is disposed on the inner surface 903 of outer layer 900 and is covered by inner layer 905.

In the embodiment of FIG. 10, sensor 300 is disposed within inner layer 905.

in the embodiment of FIG. 11, a cavity 911 is formed in inner surface 907 of inner layer 905 and sensor 300 is carried in cavity 911.

In the embodiment of FIG. 12, sensor 300 is carried on inner surface 907 of inner layer 905.

In the embodiment of FIG. 13, sensor 300 is carried on inner surface 903 of outer layer 900 and is similar to the embodiment of FIG. 9. However, apertures 913 are provided in inner layer 905 to permit more efficient transfer of heat from the patient's head.

In the embodiment of FIG. 14 sensor 300 is carried on inner surface 903 of outer layer 900 and an aperture 915 through inner layer 905 is filled with a gel.

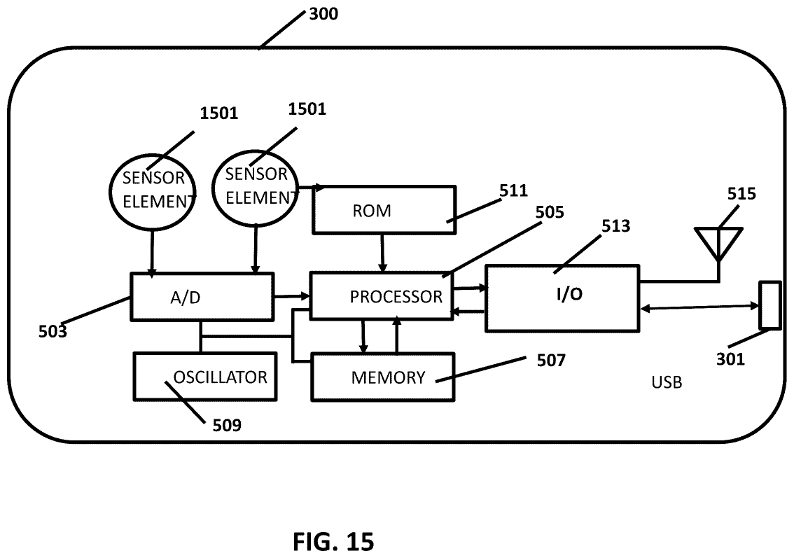

In an additional embodiment, shown in FIG. 15, two sensor elements are utilized. Sensor 300 includes a first sensing element 1501 proximate its inner surface to monitor conditions inside CRO device 100 and a second sensing element 1503 disposed proximate the outer surface of sensor 300 such that it senses conditions external to CRO device 300.

System 600 is operable to compare a rate of change or other differences between sensed parameters of sensor element 1501 and sensor element 1503. Sensor elements 1501 and 1503 may be of the same type, or sensors of different types e.g. moisture and temperature, or pressure and temperature, or combinations of other sensor elements.

Sensor 300 may have a thickness equal to the full thickness of CRO device 100, with sensing element 1501 utilized to determine ambient parameters at the outside surface external to CRO device 100 and sensing element 1503 determining parameters at the inside surface internal to CRO device 100.

By utilizing two sensing elements 1501, 1503 the differential between two parameters may be utilized rather than direct measurements. By way of example, with both sensing elements 1501, 1503 being temperature sensing elements, the temperature differential may be utilized to determine wearing time of CRO device 100.

It will be appreciated by those skilled in the art that one or more sensing elements may be used on CRO device 100 and the one or more sensing elements may sense temperature, pressure, acceleration, or other parameters. Where a plurality of sensing elements are used, they may sense the same type of parameters or different parameters and may be disposed at different locations on CRO device 100.

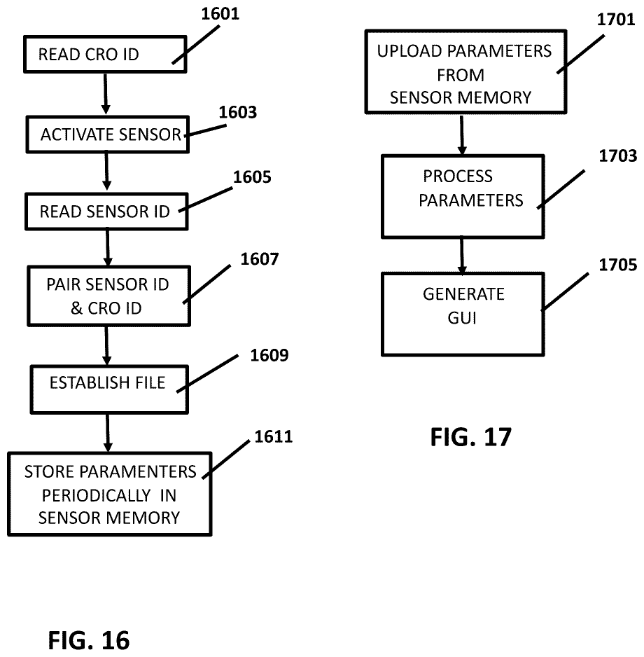

FIG. 16 is a flow diagram of the operation of sensor 300 and FIG. 17 is a generalized flow diagram of the operation of system 600.

The invention has been described in terms of various embodiments. It will be appreciated by those skilled in the art that various changes and modifications may be made to the various embodiments without departing from the scope of the invention. It is intended that the various embodiments are presented to explain the invention and to not in any way set forth limitations to the invention. It is intended that the invention is limited in scope only by the claims as presented with this application.

1. A system for determining cranial remodeling orthosis (CRO) device wearing time, comprising:
a CRO device;
said CRO device comprising at least one sensor device, said sensor device comprising a sensor memory;
an electronic device external to said CRO device;
said at least one sensor device is operable to capture specific condition parameters and to store corresponding parameter data in said sensor memory at periodic intervals;
said electronic device comprising an interface for communicating with said at least one sensor to access said parameter data stored in said sensor memory; and
a software program stored in a non-transient memory and comprising instructions executable to analyze said parameter data to determine wear times for said CRO device.

2. The system of claim 1, wherein:
said software program utilizes one or more algorithms to determine from said parameter data when specific first characteristic changes in said parameter data occur to determine when said CRO device is worn on a head and when specific second characteristic changes in said parameter data occur to determine when said CRO device is removed from said head.

3. The system of claim 1, wherein:
said software program operates on said parameter data to identify reaction rates of said sensor device to determine wear times of said CRO device.

4. The system of claim 1, comprising:
said software program executing said instructions to determine when said parameter data reaches predetermined threshold values to determine wear times of said CRO device.

5. The system of claim 1, comprising:
said software program executing said instructions to determine rapid changes in slope of said parameter data to determine wear times of said CRO device.

6. The system of claim 1, wherein:
said software program executing said instructions to identify changes in said parameter data derivatives of said parameter data, and inflection points in parameter data to determine wear times.

7. The system of claim 1, comprising:
said sensor device comprises a temperature sensor positioned on said CRO device;
said specific condition parameters are temperatures within said CRO device; and
said parameter data comprises captured temperatures.

8. The system of claim 1, wherein:
said sensor device comprises a unique sensor identification code; and
said electronic device is operable to read said unique sensor identification code.

9. The system of claim 8, comprising:
said CRO device comprises a unique CRO device identification code.

10. The system of claim 9, wherein:
said electronic device is operable to read said unique CRO device identification code.

11. The system of claim 1, wherein:
said sensor device comprises a plurality of sensor elements.

12. The system of claim 1, wherein:
said electronic device comprises one of a smart phone, tablet, and mobile computer.

13. The system of claim 1, wherein:
said electronic device comprises a set of executable instructions operable to activate said sensor when said cranial remodeling orthosis is to be initially provided to a patient.

14. The system of claim 1, comprising:
a server comprising said processor;
said electronic device in electronic communication with said processor.

15. The system of claim 1, wherein:
said electronic device comprises a graphical user interface (GUI)
said GUI selectively displays one or more of wear time for said CRO device and said specific condition parameters.

16. The system of claim 15, wherein:
said specific condition parameters are temperatures.

17. The system of claim 1, wherein:
said at least one sensor device responsive to said electronic device to become activated and to begin capturing said specific condition parameters.
